(19) United States
(12) Patent Application Publication
(10) Pub. No.: US 2016/0302967 A1
(43) Pub. Date: Oct. 20, 2016

(54) DEVICE FOR TREATING OCULAR DISEASES CAUSED BY INCREASED INTRAOCULAR PRESSURE

(71) Applicant: SAMSUNG LIFE PUBLIC WELFARE FOUNDATION, Seoul (KR)

(72) Inventor: Byung Heon AHN, Seoul (KR)

(73) Assignee: SAMSUNG LIFE PUBLIC WELFARE FOUNDATION, Seoul (KR)

(21) Appl. No.: 15/024,054

(22) PCT Filed: Sep. 23, 2014
(86) PCT No.: PCT/KR2014/008831

§ 371 Date: Jul. 6, 2016

(30) Foreign Application Priority Data
Sep. 25, 2013 (KR) 10-2013-0114037

(51) Int. Cl. A61F 9/007; A61M 27/00

(52) U.S. Cl. CPC A61F 9/00781; A61M 27/006; A61M 2210/0612

(57) ABSTRACT

One or more example embodiments relate to a device for treating ocular diseases caused by an increased intraocular pressure. The device for treating ocular diseases according to one or more example embodiments, compared to a conventional Ahmed valve implant and Baerveldt implant, does not require a donated tissue, reduces tube exposure danger, allows intraocular pressure to be easily controlled after surgery, has a slight effusion of aqueous humor to the surrounding due to a small perforated window, decreases the types and number of postoperative interventions, and decreases the number of patients with early postoperative complications.

### TECHNICAL FIELD

This application claims the benefit of Korean Patent Application No. 10-2013-0114037, filed on Sep. 25, 2013, in the Korean Intellectual Property Office, the disclosure of which is incorporated herein in its entirety by reference.

One or more example embodiments relate to a device for treating ocular diseases caused by an increased intraocular pressure.

### BACKGROUND ART

Intraocular pressure of glaucoma patients that cannot be adjusted by an intraocular pressure lowering agent is lowered by forming a detour for allowing aqueous humor in an anterior chamber of an eye to drain way under a conjunctiva. A glaucoma filtration surgery for forming a detour or a fistula to drain the aqueous humor may fail to adjust the intraocular pressure because the detour is closed after the surgery and thus the aqueous humor drainage is reduced. When the glaucoma filtration surgery should be performed due to the failure of the first surgery, the frequency of closure of the detour after the surgery may increases, and the success rate of the surgery decreases.

Depending on the type of glaucoma, that is, for refractory glaucoma such as neovascular glaucoma or secondary glaucoma associated with uveitis, the detour closure frequently occurs after the glaucoma filtration surgery, and thus the result of the surgery is not good.

Thus, when the glaucoma filtration surgery has ever failed, or when the refractory glaucoma has occurred, a surgery for installing a glaucoma drainage device is performed to prevent the detour closure and increase the success rate of the surgery. The glaucoma drainage device includes a tube configured to serve as a pathway through which aqueous humor flows out of the anterior chamber of the eye and a small disk-shaped or membrane-shaped artificial structure configured to form a space for temporarily gathering the aqueous humor under the conjunctiva and Tenon's capsule.

Since the introduction of the Molteno implant in 1968, several kinds of implants such as Krupin-Denver implant, Baerveldt implant, and Ahmed Implant have been developed and used, almost all of which use silicone as material. The implants necessarily use silicone tubes as an aqueous humor drainage pathway, and the tubes similarly have external diameters of about 640 μm and internal diameters of about 300 μm. Among these, Ahmed Implant and Baerveldt implant are most widely used.

The thickness of the tube used in these implants is significantly greater in comparison to the amount of aqueous humor drainage needed to adjust the intraocular pressure. Thus, a separate action is needed to prevent too much aqueous humor from draining out. The action includes, for example, installing a valve to control the drainage, temporarily inserting a thread into the tube during the surgery, or contracting the tube. Furthermore, when the tube that is installed under the conjunctiva is so thick that the tube may protrude toward an ocular surface, the tube may be exposed after a long time, resulting in dangerous situations. In order to prevent this, the tube should be covered with a separate tissue during the surgery.

For example, the tube may be covered with a donated hard film, pericardium, fascia, or sclera tissue.

Therefore, the present inventors tried to improve the drawbacks of conventional glaucoma drainage devices.

Throughout this specification, reference is made to several papers and patent documents, the disclosures of which are incorporated herein in its entirety by reference in order to more clearly describe the technical field to which one or more example embodiments pertain and the technical details of the present invention.

### DISCLOSURE

### Technical Problem

The present inventors tried to develop a device for treating ocular diseases caused by an intraocular pressure increased due to a decrease in drainage of aqueous humor. Eventually, the inventors manufactured a device including a membrane plate configured to contain aqueous humor, a fine tube connected to one end of the membrane plate and configured to drain the aqueous humor, the fine tube having a length of 20 to 40 mm, an internal diameter of 130 to 230 μm, and an external diameter of 250 to 400 μm, and a thread inserted into the fine tube to reach an inside of the membrane plate. After the device was used to perform a glaucoma surgery on glaucoma patients, the inventors completed one or more example embodiments by confirming the following facts: there was no need of a tissue for protecting the fine tube; the risk of exposure of the fine tube to the conjunctival surface decreased; a perforated window was so small that there was little drainage of the aqueous humor in the vicinity of the tube; the drainage of the aqueous humor was easily adjusted to appropriately adjust the intraocular pressure; and early postoperative complications such as hypotony decreased and thus the outcome of the surgery was enhanced.

Accordingly, it is an object of the one or more example embodiments to provide a device for treating ocular diseases caused by an increased intraocular pressure.

Other objects and advantages of one or more example embodiments are apparent in the following detailed description, appended claims, and accompanying drawings.

### Technical Solution

According to one or more example embodiments, a device for treating ocular diseases caused by an increased intraocular pressure includes: (a) a thin flexible membrane plate configured to contain aqueous humor and formed of a non-porous or low-porosity polymer; (b) a fine tube connected to one end of the membrane plate and configured to drain the aqueous humor to adjust intraocular pressure, the fine tube having a length of 20 to 40 mm, an internal diameter of 130 to 230 μm, and an external diameter of 250 to 400 μm; and (c) a thread inserted into an aqueous humor outlet of the fine tube to reach an inside of the membrane plate and configured to be movable back and forth in or removable from the outlet of the fine tube to adjust the amount of drainage of the aqueous humor.

The inventors tried to develop the device for treating ocular diseases caused by an intraocular pressure increased due to a decrease in drainage of aqueous humor. Eventually, the inventors manufactured a device including a membrane plate configured to contain aqueous humor, a fine tube connected to one end of the membrane plate and configured to drain the aqueous humor, the fine tube having a length of 20 to 40 mm, an internal diameter of 130 to 230 μm, and an external diameter of 250 to 400 μm, and a thread inserted into the fine tube to reach an inside of the membrane plate. After the device was used to perform a glaucoma surgery on glaucoma patients, the inventors confirmed the following facts: there was no need of a tissue for protecting the fine tube; the risk of exposure of the fine tube to the conjunctival surface decreased; a perforated window was so small that there was little drainage of the aqueous humor in the vicinity of the tube; the drainage of the aqueous humor was easily adjusted to appropriately adjust the intraocular pressure; and early postoperative complications such as hypotony decreased and thus the outcome of the surgery was enhanced.

According to a preferred embodiment of one or more example embodiments, one of the ocular diseases caused by the increased intraocular pressure may be glaucoma.

A liquid called aqueous humor is generated in an eye. The aqueous humor should be drained to the outside of the eye at a certain rate in order to maintain intraocular pressure. When there is an abnormality in the generation and drainage of the aqueous humor, the intraocular pressure increases. When the intraocular pressure increases, an optic disc is pressed, resulting in the damage to optic nerves. Depending on the degree of damage to optic nerves, the dark spot occurs in the visual field, which leads to blindness if the disease progresses to glaucoma.

According to a further preferred embodiment of one or more example embodiments, the glaucoma includes congenital glaucoma, traumatic glaucoma, glaucoma suspect, ocular hypertension, primary open-angle glaucoma, normal tension glaucoma, capsular glaucoma with pseudoexfoliation of lens, chronic simple glaucoma, low tension glaucoma, pigmentary glaucoma, primary angle-closure glaucoma, acute angle-closure glaucoma, chronic angle-closure glaucoma, intermittent angle-closure glaucoma, glaucoma secondary to eye trauma, glaucoma secondary to eye inflammation, glaucoma secondary to drugs, neovascular glaucoma, or secondary glaucoma associated with uveitis.

The device for treating ocular diseases caused by an increased intraocular pressure will be described in detail below.

The device for treating ocular diseases according to one or more example embodiments include (i) a membrane plate; (ii) a fine tube; and (iii) a thread.

(i) Membrane Plate

The device for treating ocular diseases according to one or more example embodiments include a thin flexible membrane plate configured to contain aqueous humor and made of a non-porous or low-porosity polymer. In the device for treating ocular diseases, the membrane plate is a reservoir configured to temporarily gather the aqueous humor under the conjunctiva and Tenon's capsule.

One of the most important features of one or more example embodiments is that the membrane plate included in one or more example embodiments may be flexible and thinner than a conventional implant, thus reducing a surgical incision.

The membrane plate may be made of a non-porous or low-porosity polymer. Preferably, the non-porous or low-porosity polymer may be a thin film material that is replaceable with fluoropolymer, silicone, or poly (styrene-block-isobutylene-block-styrene) (SIBS). More preferably, the fluoropolymer may be selected from a group consisting of polytetrafluorethylene, expanded polytetrafluorethylene (e-PTFE), polyhexafluoropropylene (PHFP), and perfluoroalkoxy (PFA). Most preferably, the fluoropolymer may be expanded polytetrafluorethylene (e-PTFE).

According to another preferred embodiment of one or more example embodiments, the membrane plate may have a width of 12 to 26 mm and a length of 10 to 14 mm.

(ii) Fine Tube

In addition, the device for treating ocular diseases according to one or more example embodiments include a fine tube connected to one end of the membrane plate and configured to drain the aqueous humor to adjust intraocular pressure. The fine tube has a length of about 20 to 40 mm, an internal diameter of about 130 to 230 μm, and an external diameter of about 250 to 400 μm.

As shown in FIG. 3, another one of the most important features of one or more example embodiments are that the tube through which the aqueous humor is drained has a thinner diameter than the conventional Ahmed valve implant and Baerveldt implant. The tube of the Ahmed valve implant and the Baerveldt implant has an external diameter of about 640 μm and an internal diameter of about 300 μm while the tube according to one or more example embodiments are a fine tube having an internal diameter of 130 to 230 μm and an external diameter of 250 to 400 μm.

By using the fine tube, one or more example embodiments do not require a donated tissue, reduces the tube exposure risk, allows intraocular pressure to be easily controlled after surgery, has a slight effusion of aqueous humor to the surrounding due to a small perforated window, decreases the types and number of postoperative interventions, and reduces the number of patients with early postoperative complications, as will be seen from the following embodiments.

The device for treating ocular diseases including the fine tube according to one or more example embodiments may be primarily used for a patient having a previous surgical history and also may be applied to a patient with refractory glaucoma or a patient whose surgery failed.

According to another preferred embodiment of one or more example embodiments, the fine tube may be made of silicone.

(iii) Thread

Finally, one or more example embodiments include a thread inserted into an aqueous humor outlet of the fine tube to reach an inside of the membrane plate and configured to be movable back and forth in or removable from the outlet of the fine tube to adjust the amount of drainage of the aqueous humor.

As will be seen from the following embodiments, a postoperative anterior-chamber formation intervention is even less often performed when the device for treating ocular diseases according to one or more example embodiments are used, compared to when the conventional implant is used. This is because postoperative hypotony rarely occurs due to the thread inserted into the fine tube.

It is preferable that the thread be a non-absorbable surgical suture and more preferably a non-absorbable surgical suture made of nylon or prolene.

### Advantageous Effects

The features and advantages of one or more example embodiments are summarized as follows:

(a) One or more example embodiments provide a device for treating ocular diseases caused by an increased intraocular pressure;

(b) The device for treating ocular disease according to one or more example embodiments need not cover a tube and thus does not need a donated tissue for covering the tube by using the fine tube, in comparison to the conventional implants that are used during the glaucoma surgery;

(c) The fine tube according to one or more example embodiments are so thin that the tube may not protrude or may slightly protrude toward the conjunctival surface, thus decreasing the tube exposure danger;

(d) One or more example embodiments reduce inconvenience to the patient by simply removing the thread that was inserted and maintained during a certain time of period to reach appropriate intraocular pressure. In addition, the surgery is simple because there is no need for an action to further contract the tube or reinsert the thread into the tube during the surgery, unlike the conventional implant insertion surgery;

(e) When the intraocular pressure is higher than an appropriate level after the surgery, one or more example embodiments can lower the intraocular pressure to the appropriate level by withdrawing the thread from the tube by a desired length to increase the drainage of the aqueous humor;

(f) When the conventional tube is inserted into the anterior chamber, the tube is thicker and thus the perforated window for the insertion should be larger. In this case, the aqueous humor may be unintentionally drained around the tube. However, when the find tube according to one or more example embodiments are used, the perforated window is so small that, though the aqueous humor is drained around the tube, the amount of the drainage is negligible; and

(e) The device for treating ocular diseases according to one or more example embodiments decrease the types and number of postoperative interventions and decreases the number of patients with early postoperative complications, compared to the conventional Ahmed valve implant and Baerveldt implant.

### Description of Drawings

FIG. 1 is a sectional view of a device for treating ocular diseases according to an embodiment of the present invention.

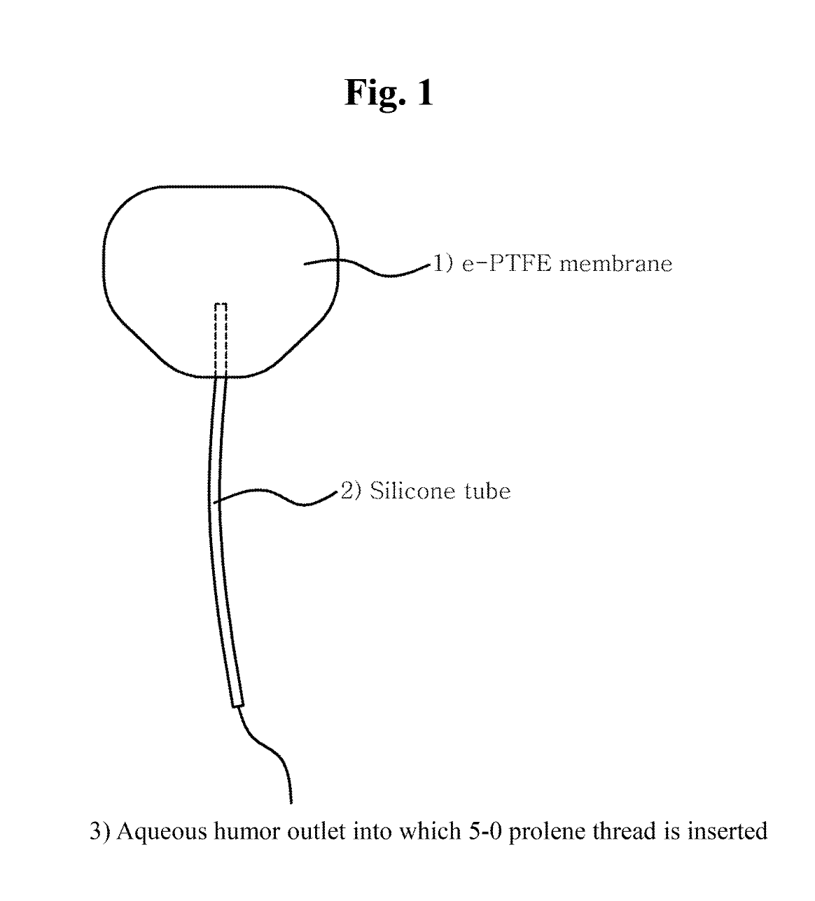

FIG. 2 is a photograph of a device for treating ocular diseases according to an embodiment of the present invention.

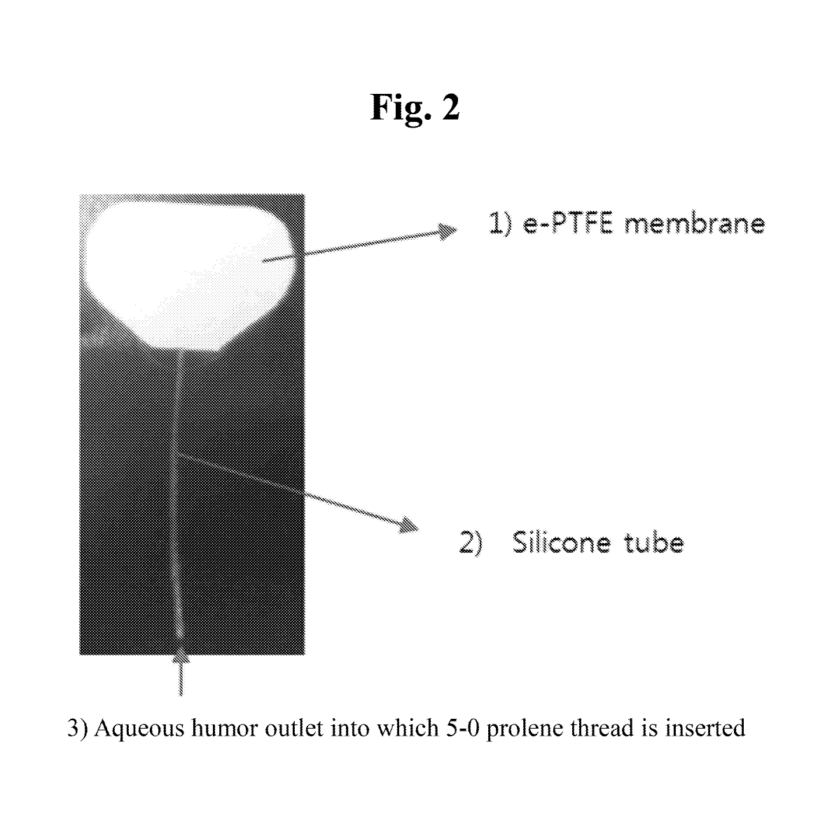

FIG. 3 shows a difference in diameter between a fine tube used in a device for treating ocular diseases according to an embodiment of the present invention and a tube used in conventional Ahmed valve implant and Baerveldt implant.

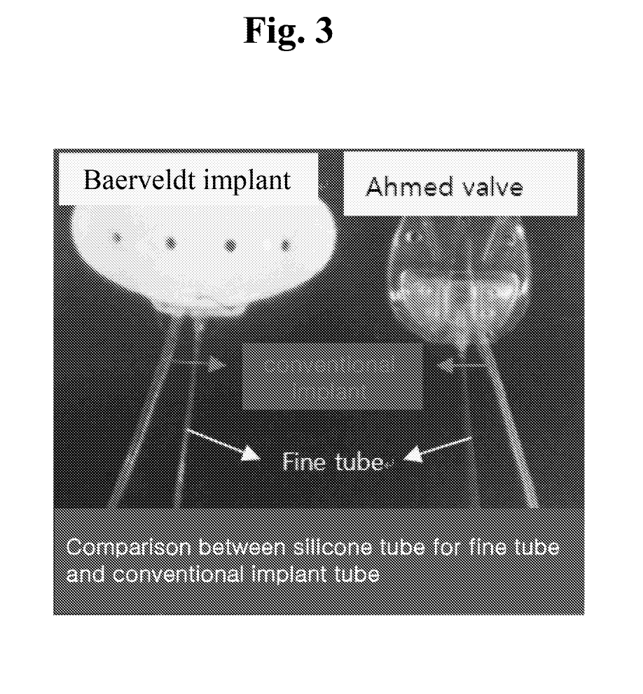

FIG. 4 is a graph showing a result of comparison in one year prognosis (i.e., intraocular pressure) after surgery between a device for treating ocular diseases according to an embodiment of the present invention and Ahmed valve implant and Baerveldt implant.

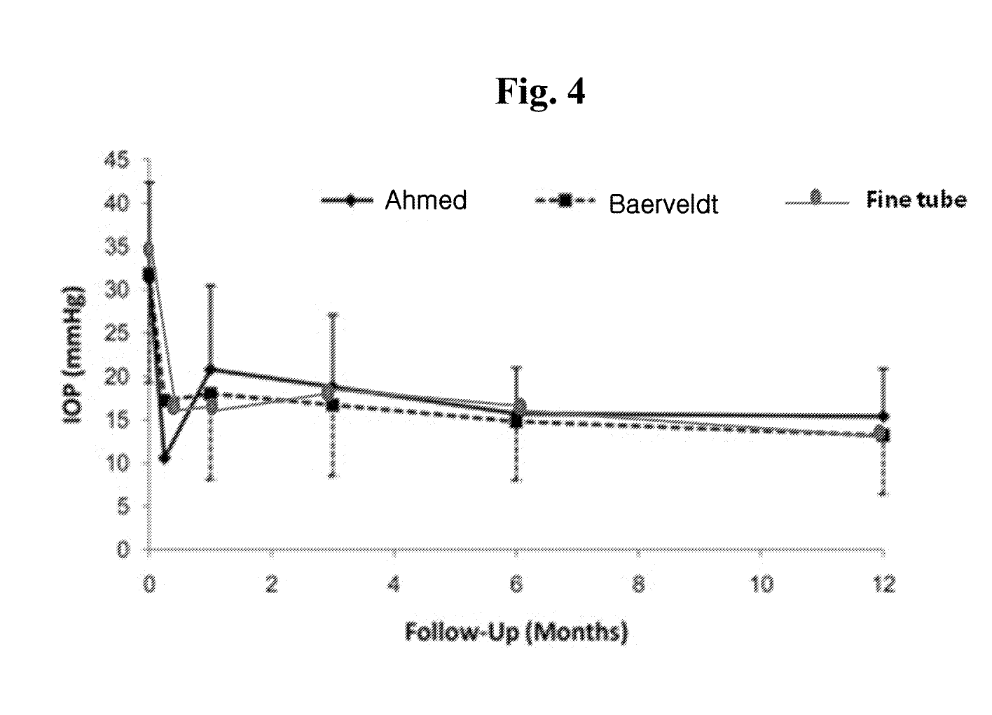

### MODES OF THE INVENTION

Hereinafter, one or more example embodiments will be described in detail with reference to embodiments. These embodiments are merely provided to explain in more detail one or more example embodiments. Accordingly, it would be obvious to those skilled in the art that one or more example embodiments are not limited thereto.

### Embodiment 1: Preparation of Glaucoma Treatment Device and Surgery Using the Same

First, an expanded polytetrafluoroethylene (e-PTFE) membrane was cut to a width of about 18 mm and a length of about 13 mm, and was shaped into an oval with an area of 180 mm² Next, a silicone tube with an internal diameter of 200 μm and an external diameter of 300 μm was inserted between double layers of the oval e-PTFE membrane and was fixed with silicone adhesive. Then the membrane was sealed at the edge with the silicone adhesive while a portion of the edge corresponding to an opposite side of the tube was not sealed so that drained aqueous humor might flow out. A 5-0 prolene thread was inserted into the fixed silicone tube, and then was pushed into an inner space of the membrane. This is to block the inside of the tube in order to prevent early postoperative hypotony.

In the department of ophthalmology at Samsung Medical Center, glaucoma surgery was performed on 30 glaucoma patients using the glaucoma treatment device prepared in this way.

### EXPERIMENTAL EXAMPLE 1

### One-Year Observation After Surgery Using Glaucoma Treatment Device

Clinical outcomes were compared for patients on whom a one-year observation after the surgery using a fine tube (that is, the glaucoma treatment device) has been performed, by using the same items as those of ABC (Ahmed Baerveldt Comparison) research result (Donald L. et al, Treatment Outcomes in the Ahmed Baerveldt Comparison Study after 1 Year of 20 Follow-up. Ophthalmology. 2011; 118:443-452), which was obtained through comparison in one-year prognosis between Ahmed valve implant and Baerveldt implant. The results were summarized in Table 1 below.

TABLE 1

| | Comparative example 1 | | Comparative example 2 | | Embodiment 1 | |
|---|---|---|---|---|---|---|
| — | (Ahmed valve group) | | (Baerveldt implant group) | | (Fine tube group) | |
| Baseline | — | | — | | — | |
| IOP (mmHg) | 31.2 ± 11.2 | | 31.8 ± 12.5 | | 35.7 ± 12.7 | |
| Glaucoma medication | 3.4 ± 1.1 | | 3.5 ± 1.1 | | 2.6 ± 0.8 | |
| No. (% of baseline) | 143 | | 133 | | 30 | |
| 1 day | — | | — | | — | |
| IOP (mmHg) | 10.0 ± 7.9 | | 18.6 ± 13.7 | | 12.0 ± 9.6 | |
| No. (% of baseline) | 142 | (99%) | 130 | (98%) | 30 | (100%) |
| 1 week | — | | — | | — | |
| IOP(mmHg) | 10.6 ± 5.6 | | 17.2 ± 12.0 | | 16.1 ± 11.3 | |
| Glaucoma medication | 0.2 ± 0.7 | | 0.9 ± 1.4 | | 0 | |
| No. (% of baseline) | 140 | (98%) | 118 | (89%) | 30 | (100%) |
| 1 month | — | | — | | — | |
| IOP(mmHg) | 20.7 ± 9.7 | | 18.0 ± 10.0 | | 13.2 ± 6.6 | |
| Glaucoma medication | 0.5 ± 1.0 | | 1.3 ± 1.5 | | 0.0 ± 0.2 | |
| No. (% of baseline) | 139 | (97%) | 130 | (98%) | 30 | (100%) |
| 3 month | — | | — | | — | |
| IOP(mmHg) | 18.8 ± 8.3 | | 16.7 ± 8.2 | | 18.0 ± 7.3 | |
| Glaucoma medication | 1.4 ± 1.3 | | 1.2 ± 1.3 | | 0.4 ± 0.6 | |
| No. (% of baseline) | 133 | (93%) | 125 | (94%) | 30 | (100%) |
| 6 month | — | | — | | — | |
| IOP (mmHg) | 15.7 ± 5.3 | | 14.8 ± 6.8 | | 16.6 ± 3.2 | |
| Glaucoma medication | 1.7 ± 1.4 | | 1.3 ± 1.3 | | 1.2 ± 0.8 | |
| No. (% of baseline) | 131 | (92%) | 125 | (94%) | 30 | (100%) |
| 1 year | — | | — | | — | |
| IOP (mmHg) | 15.4 ± 5.5 | | 13.2 ± 6.8 | | 15.6 ± 3.2 | |
| Glaucoma medication | 1.8 ± 1.3 | | 1.5 ± 1.4 | | 1.2 ± 0.8 | |
| No. (% of baseline) | 132 | (92%) | 117 | (88%) | 28 | (93%) |

As shown in Table 1, it can be seen that embodiment 1 shows a similar pattern to comparative 1 and comparative 2 as a whole in terms of the degree of adjustment of intraocular pressure (IOP) and the number of glaucoma medications used.

### EXPERIMENTAL EXAMPLE 2

### Comparison of Causes of Treatment Failure

The failure causes of the surgery in embodiment 1, comparative example 1, and comparative example 2 were summarized in Table 2 below.

TABLE 2

| | Comparative example 1 | | Comparative example 2 | | Embodiment 1 | |
|---|---|---|---|---|---|---|
| — | (Ahmed valve group) | | (Baerveldt implant group) | | (Fine tube group) | |
| Adjustment of abnormal | 9 | (6%) | 6 | (5%) | 0 | |
| IOP without additional | | | | | | |
| glaucoma surgery | | | | | | |
| Glaucoma revision surgery | 11 | (8%) | 1 | (1%) | 2 | (7%) |
| Explantation for complication | 1 | (1%) | 3 | (2%) | 0 | (0%) |
| Persistent hypotony | 0 | (0%) | 2 | (2%) | 0 | (0%) |
| No light perception | 2 | (1%) | 6 | (5%) | 0 | (0%) |
| Total | 23 | (16%) | 18 | (14%) | 2 | (7%) |

As shown in Table 2, in the conventional ABC research (i.e., comparative example 1 and comparative example 2), revision surgery performed within one year from the surgery, failure in adjustment of intraocular pressure to 21 mmHg or higher regardless of using an intraocular pressure medication, explantation of the device due to complications, persistent hypotony, and no light perception were considered as the treatment failures. In embodiment 1 (using the fine tube), the intraocular pressures of the remaining patients were adjusted with or without an intraocular pressure medication after one year, except for two patients (i.e., 7%) on whom a surgery for adjusting the position of the tube was performed because the intraocular pressure was not adjusted well or the position of the tube was not good.

### EXPERIMENTAL EXAMPLE 3

### Comparison of the Types and Number of Postoperative Interventions That are Performed on Patients

Table 3 below is a summary of the postoperative interventions that are performed on the patients, and a frequent postoperative intervention may give a burden to the patients and medical staffs and may increase the number of postoperative complications in future.

TABLE 3

| | Comparative example 1 | | Comparative example 2 | | Embodiment 1 | |
|---|---|---|---|---|---|---|
| — | (Ahmed valve group) | | (Baerveldt implant group) | | (Fine tube group) | |
| Anterior chamber reformation | 9 | (6%) | 15 | (11%) | 0 | (0%) |
| Avastin injection (NVG stratum) | 0 | (0%) | 2 | (1%) | 0 | (0%) |
| Laser revision of bleb at slit lamp | 2 | (1%) | 0 | (0%) | 0 | (0%) |
| Lase iridoplasty | No record | | No record | | 2 | (7%) |
| Needling at slit lamp | 0 | (0%) | 1 | (1%) | 0 | (0%) |
| Total number of patients treated | 10 | (7%) | 18 | (14%) | 2 | (7%) |

As shown in Table 3, a critical difference between embodiment 1 (using the fine tube) and the conventional implants (i.e., comparative example 1 and comparative example 2) is that the number of postoperative anterior chamber formation interventions in embodiment 1 is significantly smaller than that in comparative example 1 or comparative example 2. This is because postoperative hypotony rarely occurs due to a small internal diameter of the fine tube and an insertion of the thread into the fine tube in embodiment 1. The postoperative hypotony can lead to complications such as macular edema due to low intraocular pressure. The complications lasting for a long time may cause irreversible eye damage and thus cause a patient to frequently visit a hospital for the first several months after the surgery.

### EXPERIMENTAL EXAMPLE 4

### Comparison of Early Postoperative Complications

In Table 4 below, early postoperative complications were compared.

TABLE 4

| | Comparative example 1 | | Comparative example 2 | | Embodiment 1 | |
|---|---|---|---|---|---|---|
| — | (Ahmed valve group) | | (Baerveldt implant group) | | (Fine tube group) | |
| Occlusion | 3 | (2%) | 12 | (9%) | 2 | (7%) |
| Choroidal effusion | 21 | (15%) | 13 | (10%) | 0 | (0%) |
| Suprachoroidal hemorrhage | 0 | (0%) | 2 | (2%) | 0 | (0%) |
| endophthalmitis | 0 | (0%) | 1 | (1%) | 1 | (3.5%) |
| cystoid macula edema | 8 | (6%) | 2 | (2%) | 0 | (0%) |
| Shallow anterior chamber | 27 | (19%) | 26 | (20%) | 0 | (0%) |
| hypotony maculopathy | 5 | (3%) | 3 | (2%) | 0 | (0%) |
| diplopia | 9 | (6%) | 7 | (5%) | 0 | (0%) |
| cornea edema | 17 | (12%) | 29 | (22%) | 3 | (10%) |
| Tube-cornea contact | 7 | (5%) | 8 | (6%) | 0 | (0%) |
| tube erosion | 1 | (1%) | 1 | (1%) | 0 | (0%) |
| hyphema | 13 | (9%) | 22 | (17%) | 2 | (7%) |
| vitreous Hemorrhage | 2 | (1%) | 3 | (2%) | 0 | (0%) |
| Number of patients with | 61 | (43%) | 77 | (58%) | 8 | (27%) |
| early compications | | | | | | |

Choroidal effusion, shallow anterior chamber, hypotony maculopathy, and the like are complications associated with the hypotony. As shown in Table 4, it can be seen that the complications hardly occurred during the surgery according to embodiment 1 (using the fine tube). Furthermore, other important complications such as diplopia and tube erosion are caused due to the large volumes of the tube and the explant. The diplopia may dramatically drop the quality of life of these patients, and the tube erosion may induce severe complications such as infection. Thus, the tube should be necessarily covered with the conjunctiva through the revision surgery, and should be covered with a thin membrane of another part or a sclera of another person when the conjunctiva is not sufficient. Since the volumes of the tube and the explant are significantly small, the fine tube in embodiment 1 can reduce these complications, and this may be a very significant advantage.

While preferred embodiments of one or more example embodiments have been described in detail, it would be obvious to those skilled in the art that the described embodiments are merely illustrative, and one or more example embodiments are not limited thereto. Therefore, the actual scope of the present invention is to be defined by the following claims and their equivalents.

1. A device for treating ocular diseases caused by an increased intraocular pressure, the device comprising:
(a) a thin flexible membrane plate configured to contain aqueous humor and made of a non-porous or low-porosity polymer;
(b) a fine tube connected to one end of the membrane plate and configured to drain the aqueous humor to adjust intraocular pressure, the fine tube having a length of about 20 to 40 mm, an internal diameter of about 130 to 230 μm, and an external diameter of about 250 to 400 μm; and
(c) a thread inserted into an aqueous humor outlet of the fine tube to reach an inside of the membrane plate and configured to be movable back and forth in or removable from the outlet of the fine tube to adjust the amount of drainage of the aqueous humor.

2. The device of claim 1, wherein one of the ocular diseases is glaucoma.

3. The device of claim 2, wherein the glaucoma includes congenital glaucoma, traumatic glaucoma, glaucoma suspect, ocular hypertension, primary open-angle glaucoma, normal tension glaucoma, capsular glaucoma with pseudoexfoliation of lens, chronic simple glaucoma, low tension glaucoma, pigmentary glaucoma, primary angle-closure glaucoma, acute angle-closure glaucoma, chronic angle-closure glaucoma, intermittent angle-closure glaucoma, glaucoma secondary to eye trauma, glaucoma secondary to eye inflammation, glaucoma secondary to drugs, neovascular glaucoma, or secondary glaucoma associated with uveitis.

4. The device of claim 1, wherein the non-porous or low-porosity polymer is fluoropolymer, silicone, or poly(styrene-block-isobutylene-block-styrene) (SIBS).

5. The device of claim 4, wherein the fluoropolymer is selected from a group consisting of polytetrafluorethylene, expanded polytetrafluorethylene (e-PTFE), polyhexafluoropropylene (PHFP), and perfluoroalkoxy polymer (PFA).

6. The device of claim 1, wherein the membrane plate has a width of 12 to 26 mm and a length of 10 to 14 mm

7. The device of claim 1, wherein the fine tube is made of silicone.

8. The device of claim 1, wherein the thread is a non-absorbable surgical suture.

9. The device of claim 8, wherein the non-absorbable surgical suture is made of nylon or prolene.
